SEQUENCE LISTING (27 sequences)

SEQ ID NO: 1 - protein, 5 aa, Mus musculus
NYAMS

SEQ ID NO: 2 - protein, 17 aa, Mus musculus
TISTGGSSTFYPDSVKG

SEQ ID NO: 3 - protein, 9 aa, Mus musculus
HAYEAWFAY

SEQ ID NO: 4 - protein, 10 aa, Mus musculus
SASSSVSYMY

SEQ ID NO: 5 - protein, 7 aa, Mus musculus
LTSTLAS

SEQ ID NO: 6 - protein, 9 aa, Mus musculus
QQWNSHPLT

SEQ ID NO: 7 - protein, 5 aa, Mus musculus
GYVMS

SEQ ID NO: 8 - protein, 16 aa, Mus musculus
SISSGGSSYYSDRVKG

SEQ ID NO: 9 - protein, 8 aa, Mus musculus
GYNYAMDY

SEQ ID NO: 10 - protein, 11 aa, Mus musculus
KASQDVGTAVA

SEQ ID NO: 11 - protein, 7 aa, Mus musculus
WASTWHT

SEQ ID NO: 12 - protein, 9 aa, Mus musculus
QQYKLFPLT

SEQ ID NO: 13 - protein, 118 aa, Mus musculus
EVMLVESGGGLVKPGGSLKLSCAASGFAFSNYAMSWIRQIPEKRLEWVATISTGGSSTFYPDSVKGRFTISRDNAKNTLYLQMSSLRSEDTAMYYCTRHAYEAWFAYWGQGTLVTVSA

SEQ ID NO: 14 - protein, 106 aa, Mus musculus
QIVLTQSPALMSASPGEKVTMTCSASSSVSYMYWYQQRPRSSPKPWIYLTSTLASGVPTRFSGSGSGTSYSLTISSMEAEDAATYYCQQWNSHPLTFGSGTKLEIK

SEQ ID NO: 15 - protein, 116 aa, Mus musculus
EVKLVESGGGLVKPGGSLKLSCAASGFTFSGYVMSWVRQTPDKRLEWVASISSGGSSYYSDRVKGRLTISRDNARNILFLQMSSLRSEDSAMYYCANGYNYAMDYWGQGTSVTVSS

SEQ ID NO: 16 - protein, 107 aa, Mus musculus
DIVMTQSHKFMSISVGDRVSITCKASQDVGTAVAWYQQKPGQSPQLLIYWASTWHTGVPDRFTGSGSGTDFTLTITNVQSEDLADYFCQQYKLFPLTFGAGTRLELK

SEQ ID NO: 17 - DNA, 354 bp, Mus musculus
gaagtgatgctggtggagtctgggggaggcttagtgaagcctggagggtccctgaaactctcctgtgcagcctctggatttgctttcagtaattatgccatgtcctggattcgccagattccggagaagaggctggagtgggtcgcaaccattagtactggtggtagttccaccttctatccagacagtgtgaaggggcgattcaccatctccagagacaatgccaagaataccctatacctgcaaatgagcagtctgaggtctgaggacacggccatgtattactgtacaagacatgcctacgaggcctggtttgcttactggggccaagggactctggtcactgtctctgca

SEQ ID NO: 18 - DNA, 318 bp, Mus musculus
caaattgttctcacccagtctccagcactcatgtctgcatctccaggggagaaggtcaccatgacctgcagtgccagctcaagtgtaagttacatgtactggtaccaacagaggccaagatcctcccccaaaccctggatttatctcacatccaccctggcttctggagtccctactcgcttcagtggcagtgggtctgggacctcttactctctcacaatcagcagcatggaggctgaagatgctgccacttattactgccagcagtggaatagtcacccactcacgttcggctcggggacaaagttggaaataaaa

SEQ ID NO: 19 - DNA, 348 bp, Mus musculus
gaagtgaagctggtggagtctgggggaggcttagtgaagcctggagggtccctgaaactctcctgtgcagcctctggattcactttcagtggctatgtcatgtcttgggttcgccagactccagacaagaggctggagtgggtcgcatctattagtagtggtggtagttcctattattcagatagagtgaagggccgactcaccatctccagagataatgcccggaacatcctgttcctgcaaatgagtagtctgaggtctgaggactcggccatgtattactgtgcaaacggctacaactatgctatggactactggggtcaagggacctcagtcaccgtctcctca

SEQ ID NO: 20 - DNA, 321 bp, Mus musculus
gacattgtgatgacccagtctcacaaattcatgtcaatatcagtaggcgacagggtcagcatcacctgcaaggccagtcaggatgtgggtactgctgttgcctggtatcaacagaaacccggacaatctcctcaactactgatttactgggcatccacctggcacactggagtccctgatcgcttcacaggcagtggatctgggacagatttcactctcaccattaccaatgtgcagtctgaagacttggcagactatttctgtcagcaatataaactctttccgctcacgttcggtgctgggaccaggctggagctgaaa

SEQ ID NO: 21 - protein, 21 aa, Homo sapiens
EQPQVELFVKAGSDGAKIGNC

SEQ ID NO: 22 - protein, 4 aa, Homo sapiens
VELF

SEQ ID NO: 23 - protein, 9 aa, Artificial Sequence
  synthetic peptide
GEQPQVELF

SEQ ID NO: 24 - protein, 14 aa, Artificial Sequence
  synthetic peptide
SGSGSGEQPQVELF

SEQ ID NO: 25 - protein, 9 aa, Artificial Sequence
  synthetic peptide
GSGSGSGSG

SEQ ID NO: 26 - protein, 5 aa, Homo sapiens
QVELF

SEQ ID NO: 27 - protein, 6 aa, Homo sapiens
QVELFV

(19) United States
(12) Patent Application Publication
(10) Pub. No.: US 2024/0002499 A1
(43) Pub. Date: Jan. 4, 2024

(54) ANTI-CLIC1 PROTEIN ANTIBODIES AND THE DIAGNOSTIC AND THERAPEUTIC USES THEREOF

(71) Applicant: Universita' Degli Studi di Milano, Milano (IT)

(72) Inventors: Michele Mazzanti, Genova (IT); Valentina Carlini, Bussero (MI) (IT); Ivan Verducci, Gorgonzola (MI) (IT); Francesca Cianci, Foggia (IT); Gaetano Cannavale, Lodi (LO) (IT)

(73) Assignee: Universita' Degli Studi di Milano, Milano (IT)

(21) Appl. No.: 18/253,940

(22) PCT Filed: Nov. 26, 2021
(86) PCT No.: PCT/EP2021/083126

§ 371 Date: May 23, 2023

(30) Foreign Application Priority Data
Nov. 27, 2020 (IT) 102020000028754

(51) Int. Cl. C07K 16/28; A61P 35/00

(52) U.S. Cl. CPC C07K 16/28; A61P 35/00; C07K 2317/76; C07K 2317/34; A61K 2039/505

(57) ABSTRACT

Disclosed are an antibody able to recognise and specifically bind the CLIC1 protein located on the cell membrane and inhibit its ion channel function; the uses of said antibody in the diagnostic and therapeutic fields; and pharmaceutical compositions containing the antibody.

The present invention relates to an antibody able to recognise and specifically bind the “Chloride intracellular channel 1” (CLIC1) protein located on the cell membrane, inhibiting its ion channel function. The invention also relates to the uses of the anti-CLIC1 antibody in the diagnosis and treatment of disorders associated with CLIC1 activation, especially tumours, neurodegenerative disorders and inflammatory states. The invention also provides pharmaceutical compositions containing the antibody for use in the diagnostic and therapeutic fields.

### BACKGROUND TO THE INVENTION

Some solid tumours, especially glioblastoma and the more aggressive forms of colorectal cancer, are still associated with high mortality because of the lack of treatments able to eradicate them effectively. Current treatments, ranging from surgery to radio- and chemotherapy, do not generally provide a permanent cure, and cases of recurrent processes are preponderant. There is a widespread opinion that the recurrence of said tumours is attributable to the presence in the tumour mass of a minority cell population known as cancer stem cells. Conventional treatments are effective against the tumour mass, but cancer stem cells are resistant.

Various studies report that the cells of some solid tumours accumulate chloride ions and undergo a substantial reduction in volume when said ions exit as a result of an osmotic outflow of water. The cells consequently acquire an elongated shape, and this phenomenon has proved to be crucial to cell division and migration.

Overexpression of CLIC1 has recently been observed in various solid human tumours compared with normal tissue, suggesting the potential involvement of CLIC1 in tumour origin.

CLIC1 protein belongs to the small category of metamorphic proteins. Two entirely separate forms thereof exist: a cytoplasmic form and a transmembrane form. Although the hydrophilic structure of CLIC1 is known, the structure of the membrane protein (tmCLIC1) has never been resolved [1, 2]. CLIC1 is known to undergo a structural transformation, with formation of a dimer, under stress conditions [1]. Under these conditions, it binds to the inner membrane. Morphofunctional studies demonstrate that the next step is insertion into the double lipid layer of the dimeric structure [1-5]. The final structure acquired by CLIC1 in the membrane is not yet known. Two dimers probably aggregate in a quaternary structure, but there is also a possibility that the single dimer is still functional, or that multiple dimers aggregate. There are several reasons why the structure of tmCLIC1 has never been resolved. Firstly, a very limited amount of protein colonises the membrane, even under the most favourable conditions. Moreover, when an attempt is made to isolate the transmembrane form, the protein loses its membrane location, and probably also its quaternary structure. The characterisation of tmCLIC1 has been conducted over the years by methods such as electrophysiology and dynamic microscopy. One of the aspects which has helped to clarify the role and some structural characteristics of the membrane protein is its ability to act as a chlorine-permeable ion channel [4, 5].

The transmembrane form of CLIC1 is specifically present in cells which are in a state of chronic activation. tmCLIC1 is particularly present in solid tumours. In glioblastoma and the metastatic forms of colon cancer in particular, tmCLIC1 plays a key role in proliferative, migratory and tissue infiltration processes.

Inhibition of tmCLIC1 activity with the specific IAA94 blocker, or reduction of the expression of the protein in the cell using RNA interference techniques, exhibits a drastic slowing in the proliferation of the tumour cells, both in vitro and in vivo. The tmCLIC1 inhibitor currently used in in vitro experiments, namely IAA94, is unusable in vivo for its side effects.

From both the diagnostic and therapeutic standpoints, in order to counteract the growth and invasion of solid tumours it is necessary to develop highly specific diagnostic and therapeutic elements able to intercept tmCLIC1 without any side effects.

### DESCRIPTION OF THE INVENTION

The invention provides an antibody which recognises and binds the CLIC1 protein located on the plasma membrane and inhibits its function, thereby allowing various pathological conditions wherein the protein is functionally expressed in the plasma membrane to be identified and treated (tmCLIC1).

The invention therefore relates to an antibody which is directed against the extracellular domain of the CLIC1 protein located on the plasma membrane, and which inhibits its ion channel function. In particular, by using electrophysiology techniques in whole-cell configuration on single cell in primary cultures of glioblastoma or colorectal cancer cell lines, it has been found that the antibody inhibits at least 90% of the chloride ion current compared with the inhibition of the same ion channel by the IAA94 blocker at a concentration of 100 μM, by binding to the extracellular portion of tmCLIC1.

Epitope mapping studies demonstrate that a region of the CLIC1 transmembrane protein recognised by an antibody able to inhibit its ion channel function contains the amino acid sequence QVELF.

Antibodies able to bind CLIC1 protein in the membrane and inhibit its ion channel function have been isolated, and their structure determined.

In one embodiment of the invention, the anti-CLIC1 membrane protein antibody comprises a heavy chain variable domain (VH) and a light chain variable domain (VL), both of which domains in turn comprise three complementary determining regions (CDRs), wherein:
VH CDR1, CDR2 and CDR3 comprise or consist of the sequences SEQ ID NO:1, SEQ ID NO:2 and SEQ ID NO:3 respectively, or sequences at least 90%, preferably at least 95%, identical thereto;
and
VL CDR1, CDR2 and CDR3 comprise or consist of the sequences SEQ ID NO:4, SEQ ID NO:5 and SEQ ID NO:6 respectively, or sequences at least 90%, preferably at least 95%, identical thereto;
or
VH CDR1, CDR2 and CDR3 comprise or consist of the sequences SEQ ID NO:7, SEQ ID NO:8 and SEQ ID NO:9 respectively, or sequences at least 90%, preferably at least 95%, identical thereto;
and
VL CDR1, CDR2 and CDR3 comprise or consist of the sequences SEQ ID NO:10, SEQ ID NO:11 and SEQ ID NO:12 respectively, or sequences at least 90%, preferably at least 95%, identical thereto.

In a preferred embodiment of the invention, the antibody comprises:
a heavy chain variable domain (VH) which comprises or consists of the sequence SEQ ID NO:13, or a sequence at least 90%, preferably at least 95%, identical thereto;
and
a light chain variable domain (VL) which comprises or consists of SEQ ID NO:14, or a sequence at least 90%, preferably at least 95%, identical thereto;
or
a heavy chain variable domain (VH) which comprises or consists of the sequence SEQ ID NO:15, or a sequence at least 90%, preferably at least 95%, identical thereto;
and
a light chain variable domain (VL) which comprises or consists of SEQ ID NO:16, or a sequence at least 90%, preferably at least 95%, identical thereto.

Preferably, the antibody is an immunoglobulin of class G and subclass IgG1, IgG2, IgG3 or IgG4. As well as comprising the variable regions of the light and heavy chains, the antibody according to the invention can also comprise a constant region of the light chain containing a CL domain, and a constant region of the heavy chain containing the domains CH1-CH3 and a “hinge” region, optionally modified.

The antibody can also be humanised, ie. can comprise amino acid sequences or residues of human and non-human origin. For example, the humanised antibody can comprise one or both variable domains wherein all the hypervariable regions (CDR) correspond to those of a non-human antibody, while the “framework” regions correspond to those of a human antibody. The humanised antibody can comprise at least one portion of a constant region of human derivation. In general, a “humanised” form of the antibody relates to an antibody which has undergone a process of humanisation. Methods for humanising an antibody are known in the art (see, for example, Almagro J. C. and Fransson J., (2008) Frontiers in Bioscience 13: 1619-1633), and are based on methods such as CDR-grafting, Resurfacing, Superhumanisation and Human String Content Optimisation.

Another aspect of the invention relates to a fragment of the anti-CLIC1 antibody described herein, wherein said fragment is selected from Fab, Fab′, Fab′-SH, Fv, scFv, (Fab′)2, diabodies (dAb) and single-domain antibody (sdAb).

A further aspect of the invention relates to a molecule of nucleic acid encoding the variable regions or light and heavy chains of the anti-CLIC-1 antibody. In preferred embodiments:
the molecule of nucleic acid encoding the heavy chain variable region consists of the sequence SEQ ID NO:17, or of a sequence at least 90%, preferably at least 95%, identical thereto;
and
the molecule of nucleic acid encoding the light chain variable region consists of the sequence SEQ ID NO:18, or of a sequence at least 90%, preferably at least 95%, identical thereto;
or
the molecule of nucleic acid encoding the heavy chain variable region consists of the sequence SEQ ID NO:19, or of a sequence at least 90%, preferably at least 95%, identical thereto;
and
the molecule of nucleic acid encoding the light chain variable region consists of the sequence SEQ ID NO:20, or of a sequence at least 90%, preferably at least 95%, identical thereto.

Further aspects of the invention relate to an expression vector containing a nucleic acid encoding the antibody and the parts or fragments thereof, and a host cell containing the expression vector.

Another aspect of the invention relates to a conjugated molecule containing an antibody according to the present invention covalently bonded to a compound selected from isotopic or fluorescent marker; toxin; enzyme; medicament.

A further aspect of the invention relates to a pharmaceutical or diagnostic composition containing the antibody described herein. The composition comprises pharmaceutically acceptable carriers and excipients, such as diluents, adjuvants, buffering agents, emulsifiers, humectants, solubilisation agents or other substances which allow or facilitate the administration of the antibody and its distribution in the body and delivery to the site of action, or which reduce its toxicity, increase its bioavailability or promote compliance by the individual to whom it is administered. For the selection of an excipient suitable for the applications specified herein, see the manual “Handbook of Pharmaceutical Excipients”, 5th Edition, R. C. Rowe; P. J. Seskey and S. C. Owen, Pharmaceutical Press, London, Chicago. The composition can take the form of a solution, suspension, emulsion, tablet, capsule, microcapsule, liposome, powder, sustained-release formulation or lyophilizate. Suitable methods of administration include oral, sublingual, nasal and parenteral administration, and in particular intravenous, subcutaneous, intramuscular, intradermal and intrathecal administration.

The anti-CLIC1 protein antibody located on the plasma membrane can be used in diagnostic or therapeutic applications.

Its diagnostic applications include determination and monitoring of neurodegenerative processes and tumours. In general, the diagnostic applications are based on determination of the CLIC1 protein content in the membrane via the bond with the antibody according to the invention, for example using immunohistochemical, immunoenzymatic or immunoradiometric methods, such as radioimmunoassays, immunofluorescence assays or immunoenzymatic assays.

A typical diagnostic application involves determining the presence or monitoring the progress of chronic neurodegenerative processes such as Alzheimer's disease. Murine models have demonstrated that the tmCLIC1 protein already exhibits a significant increase at the pre-symptomatic stages of the disorder. The circulating or peripheral monocytes are immediately activated on the onset of inflammatory processes affecting the central nervous system. In the case of Alzheimer's disease, the circulating monocytes are recruited by pro-inflammatory signals originating from the microglial cells, which represent the brain's intrinsic immune system. Said monocyte activation process rapidly expands to the entire circulatory system, enabling the focus of the inflammation to be identified by using the antibody according to the invention on a sample of peripheral blood to determine the CLIC1 protein in the membrane of the monocytes. A significant increase in the antibody signal detected at different times is indicative of chronic inflammation, and will necessitate further and more in-depth tests, such as cerebrospinal fluid collection, or a CAT or MRI scan. Once isolated, the patient's monocytes can be analysed for their bond to the antibody by spectrophotometry to determine the intensity of fluorescence. More detailed cell analyses can be conducted using flow cytofluorometry and various microscopy techniques, ranging from polarised light to confocal technology.

Also in the case of tumours, detection of CLIC1 on the surface of the circulating monocytes over a period of time is a useful indicator of the presence of cancer, allowing further and more specifically-targeted tests to be conducted to identify the presence and location of abnormalities.

From the therapeutic standpoint, the experiments demonstrate that the antibody inhibits the operation of the ion channel formed by the tmCLIC1 protein, with repercussions on the activation of the circulating monocytes and microglial cells, and on the proliferation, migration and production of ROS by the microglial and tumour cells. Said experimental findings demonstrate the usefulness of the antibody for the intended therapeutic applications, which mainly include treatment of tumours wherein CLIC1 protein is present in the membrane, especially solid tumours such as glioblastoma and colon, pancreas, prostate and breast cancer; neurodegenerative processes including, in particular, Alzheimer's disease, and the inflammatory states present in various disorders. For the treatment of disorders affecting the central nervous system, the antibody or a fragment thereof could be administered directly in situ or via delivery systems able to cross the blood-brain barrier. For example, T lymphocyte cells engineered with the CarT technique could be used to convey the action of the antibody into the central nervous system. Said engineered cells permeate the blood-brain barrier and recognise the activated microglial cells responsible for neurodegenerative symptoms or, alternatively, glioblastoma tumour cells, and bind to the CLIC1 protein, thereby inhibiting its activation, and reducing its harmful effects.

### DESCRIPTION OF FIGURES

FIG. 1. Spatial distribution of CLIC1 protein in microglial cells in control (left) and after addition of 10 μM of 70% hydrogen peroxide (H₂O₂). The CLIC1 protein migrates as a result of oxidation, and accumulates at the membranes.

FIG. 2. Location of CLIC1 protein in microglial cells. (A) Control cells fixed, permeabilised and labelled with a commercial antibody against the complete CLIC1 protein. (B) Use of the monoclonal antibody tmCLIC1omab in resting microglial cells in vivo. (C) Microglial cells in vivo following exposure to β-amyloid.

FIG. 3. Growth curves of cultured resting microglial cells (circles), after stimulation with β-amyloid (squares) and in the presence of amyloid peptide and tmCLIC1omab monoclonal antibody.

FIG. 4. Electrophysiology experiments on BV2 microglial cells. Recordings of membrane currents with the patch-clamp technique in whole-cell configuration. From top left: family of currents promoted by potential steps from −60 to +80 mV, in control, in the presence of amyloid peptide, after perfusion of the antibody and following further perfusion of the tmCLIC1 ion-channel blocker.

FIG. 5. Production of ROS in cultured microglial cells in control cultures (baseline), after addition of β-amyloid and tmCLIC1omab antibody. In the presence of the antibody, ROS production falls to the control level.

FIG. 6. Human monocytes in vivo hybridised with tmCLIC1omab antibody. (A and B) Monocytes isolated from the blood of Alzheimer's disease patients. (C and D) Monocyte cells from a healthy individual in the presence of the same antibody.

FIG. 7. Two examples of human glioblastoma cells isolated and hybridised in vivo with the tmCLIC1omab antibody. Distribution mainly takes place in the membrane.

FIG. 8. Growth curves of human glioblastoma cells in vitro under control conditions (squares) and in the presence of tmCLIC1omab antibody (circles) in a 4-day period.

FIG. 9. (A) Western blot of control cells and knockout cells for CLIC1 protein. (B) Growth curves of control glioblastoma cell cultures and glioblastoma cell cultures treated with tmCLIC1omab and IAA94. (C) Knockout tumour cells for CLIC1 (C/ic/′) in control and in the presence of the antibody and IAA94.

FIG. 10. Relationship between current density and voltage of CLIC1-mediated chloride current, measured by the perforated patch clamp technique. The cells of three different primary human tumour cultures (glioblastoma, colorectal and prostate) exhibit a CLIC1-mediated current (grey circles, squares and triangles) which is almost totally eliminated by perfusion of the tmCLIC1omab antibody (black circles, squares and triangles).

FIG. 11. Production of ROS in human glioblastoma cells in primary culture under control conditions treated with IAA94 or tmCLIC1omab antibody. In the presence of the antibody and the tmCLIC1 blocker, ROS production falls about fourfold.

FIG. 12. Magnetic resonance scan of brains of immunodepressed mice two months after injection of glioblastoma stem cells into the brain. A: control condition. B: condition wherein the cells were pre-treated with anti-tmCLIC1 antibody.

FIG. 13. Colorectal cancer cell line COLO201spheroids treated with IAA94 or tmCLIC1omab antibody from 24 to 96 hours. Spheroids' area in the presence of CLIC1 inhibitors is significantly smaller compared to the control from 48 h (One-way ANOVA; ****CTR vs IAA94; p=0.0001, n=20; ****CTR vs anti-NH2; p=0.0001, n=22). Scale bar: 20 uM

FIG. 14. Trans well assay for evaluation of migration (right) and invasion (left) potential of COLO 201 WT cells treated with IAA94 and tmCLIC1omab antibody (black dots), and knockdown cells (clear dots). COLO201 cells demonstrate a significant lower number of migrative nuclei in presence of both IAA94 and tmCLIC1omab compared to control (One-way ANOVA, CTR vs IAA94****, p<0.0001; CTR vs Anti-NH2****, p<0.0001, Tukey's multiple comparison test n=16; SCR vs shl ****, p<0.0001; SCR vs sh3****, p<0.0001, Tukey's multiple comparison test n=13). Invasion assay on COLO201 cells show the same behavior seen for migration assay (One-way ANOVA, CTR vs IAA94****, p<0.0001; CTR vs Anti-NH₂****, p<0.0001, Tukey's multiple comparison test n=16; SCR vs shl ****, p<0.0001; SCR vs sh3****, p<0.0001, Tukey's multiple comparison test n=14). On the bottom is reported a representative picture of the membranes after 72 h of treatments. Scale bar: 20 uM

FIG. 15. Western blot analysis of MAP38 in its total and activated phosphorylated form and Metalloproteinase 7 in COLO201 after 72 h of treatment with IAA94 and monoclonal antibody. It is visible that the expression of pMAP38 is downregulated in the presence of both treatments, and the expression of MMPI is mainly abolished in the presence of IAA94 and tmCLIC1omab.

FIG. 16. Comparison between polyclonal antibody and tmCLIComab growth glioblastoma cells inhibition. The panel on the left show the action of the polyclonal antibody compared to IAA94 on cell growth over 96 hours experiments. On the right a similar experiment but using tmCLIC1omab. An additional information is that the percentage of cell death exposing cells for 96 hours was 65% for the polyclonal antibody trial and only 15% for tmCLIComab, the latter similar to the IAA94 action.

FIG. 17. Current/voltage relationship of CLIC1 membrane current in whole-cell patch clamp experiments. HEK cells were transfected with CLIC1 plasmid to overexpress the CLIC1 protein. After recording control current (filled cycles; n=5) cells were perfused alternatively with the polyclonal antibody (open triangles; n=7), tmCLIComab (inverse open triangles; n=10) and with 50 μM IAA94 (filled squared; n=11). The whole cells current is expressed as a current density.

### DETAILED DESCRIPTION OF THE INVENTION

The activity and biological effects of the antibody according to the invention have been studied in various experimental models. Said studies used an IgG1 class antibody containing the heavy chain variable region identified as SEQ ID NO:13 and the light chain variable region identified as SEQ ID NO:14 (hereinafter called “tmCLIC1omab”). Similar results were obtained with a class IgG2b antibody containing the heavy chain variable region identified as SEQ ID NO:15 and the light chain variable region identified as SEQ ID NO:16.

Effects of the tmCLIC1omab antibody in neurodegenerative processes.

Processes specific to the central nervous system (CNS) involving chronic activation of the microglia, the intrinsic immune system of the brain, were studied. Neurodegenerative syndromes such as Alzheimer's disease are considered to be of sporadic origin in 95% of cases, i.e. where the origin is non-genetic, and in the majority of cases unknown. These are irreversible processes which lead to rapid degeneration of the CNS and, in particular, to the death of groups of neurones. There are many theories about the factors responsible for the onset of said disorders. The immune system definitely plays a crucial role. The microglia, which chronically activates and acts on very large areas of the brain, seems to be mainly responsible for neurone death. The mechanism appears to be comparable to an autoimmune reaction. With advancing age and/or under particular conditions of oxidative stress, a significant increase in β-amyloid peptide produced by the nerve cells seems to take place. The function of said peptide is currently unknown. However, an increase in the concentration thereof is known to trigger protein aggregation, with the formation of oligomers. Said aggregated species are currently considered to be among the most toxic to the neurones. In addition to oligomers, the more complex aggregations give rise to the formation of fibres, and eventually, β-amyloid plaques. The microglia plays a very complex part in this situation. Primarily, it is activated chronically during oligomer formation, recognises oligomers as foreign elements, and attacks them. Microglial cells then release pro-inflammatory substances such as TNF-α, ROS, NO, IL-1β, etc. Said substances, which are useful to counteract bacterial infections locally, become toxic to huge populations of neurones if released indiscriminately. Together with this action the microglia, by releasing a series of cytokines, alerts the entire body's immune system, recruiting the circulating monocytes to the CNS; said monocytes cross the blood-brain barrier and, in their capacity as macrophages, support the microglia in its action. This scenario makes it possible to intercept the signal of a chronic inflammation or a chronic state of microglial activation in the circulatory system. What is required is identification of a molecular marker which can be recognised in the circulating monocytes. The characterisation of said blood cells in healthy volunteers and patients with Alzheimer's disease was the subject of a recent publication [6].

The tmCLIC1omab antibody was used in immunofluorescence experiments on monocytes isolated from peripheral blood, in electrophysiological experiments designed to demonstrate their blocking activity against the functionality of membrane CLIC1, and in experiments designed to demonstrate how the antibody inhibits ROS production following stimulation of the microglial cells with β-amyloid.

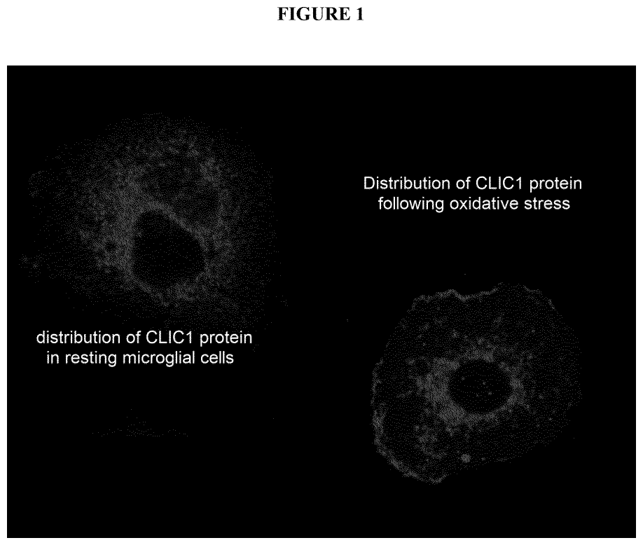

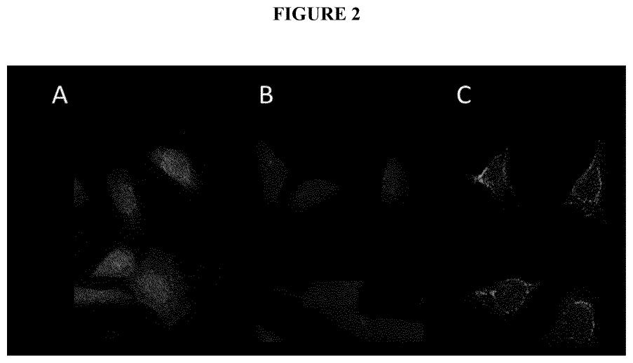

In resting microglial cells, CLIC1 protein is widely distributed in the cytoplasm, as shown in FIG. 1. The same figure shows the distribution of the protein after 30 minutes of stimulation with a strongly oxidising compound (H₂O₂ 10 μM). The migration of the protein to the plasma membrane gives rise to insertion of CLIC1 as transmembrane protein. The tmCLIC1omab antibody was used on live cells to specifically identify tmCLIC1. FIG. 2 shows an experiment wherein microglial cells were fixed and permeabilised and CLIC1 was marked with a commercial antibody against the whole CLIC1 protein (A). In live cells, the tmCLIC1omab antibody was used before (B) and after (C) stimulation with β-amyloid. It is evident that tmCLIC1omab only reacts from the exterior, identifying the CLIC1 protein after stimulation by microglial activation.

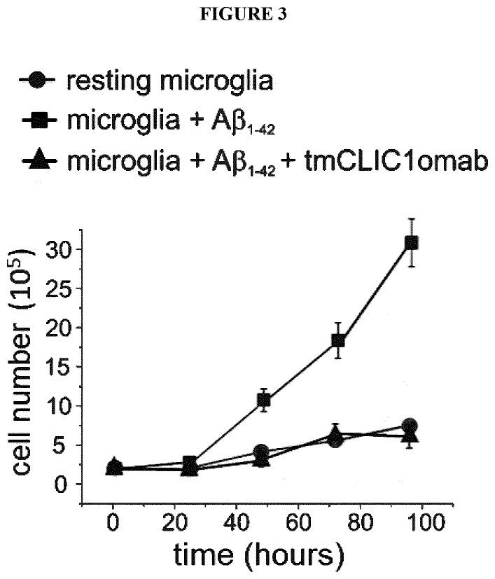

One of the reactions of the microglial cells that follow stimulation with amyloid peptide is intense proliferation. FIG. 3 shows a growth curve of microglial cells in resting conditions during a 4-day observation period. The addition of 1 μM of β-amyloid gives rise to cell proliferation with an exponential trend. The action of the anti-tmCLIC1 monoclonal antibody demonstrates that despite the presence of amyloid, cell proliferation is completely inhibited.

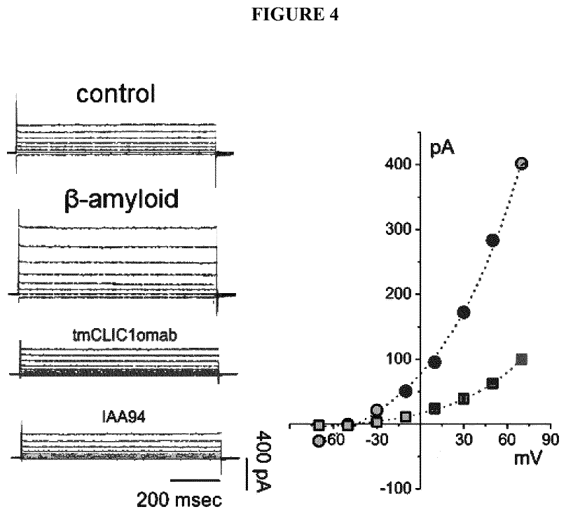

Functional electrophysiology experiments gave a similar result. FIG. 4 shows a patch-clamp experiment in whole-cell configuration wherein the BV2 microglial cells exhibit a membrane current that is considerably increased by the addition of β-amyloid peptide. Treatment with tmCLIC1omab reduces said current about fourfold. A further treatment with the specific ion channel blocker formed by tmCLIC1 only generates a minimal decrease in current. The current-to-voltage ratio (on the right) better quantifies the reduction in current.

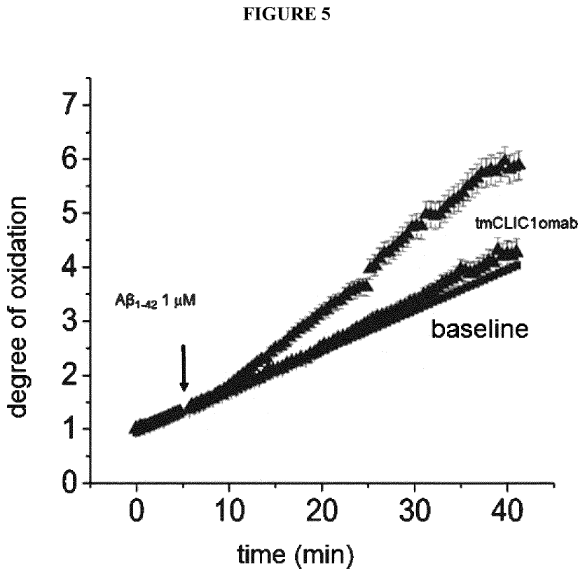

A second functional experiment performed on microglial cells involved measuring the production of reactive oxygen species (ROS), which contribute to creating a highly oxidising environment harmful to the neurones. Once again (FIG. 5), a physiological increase in cytoplasmic ROS was recorded (baseline), with ROS production after addition of β-amyloid, and finally, cell oxidation during the simultaneous presence of amyloid peptide and the tmCLIC1omab antibody.

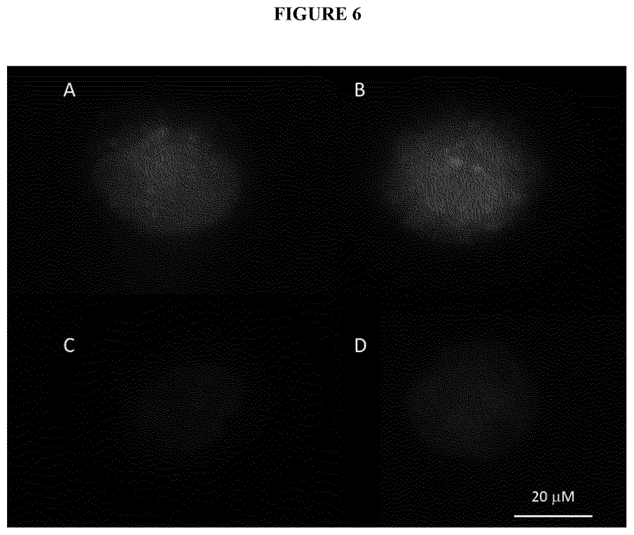

In the final experiment, the monoclonal antibody was used on monocytes isolated from the peripheral blood of patients with a diagnosis of Alzheimer's disease and controls (for full details, see [6]). FIG. 6 shows two single monocytes in each condition treated by a similar procedure. In all cases, the monocytes were first incubated with tmCLIC1omab, and then with a fluorescent secondary antibody. The images in FIG. 5 demonstrate the specificity of the antibody for the membrane protein.

Effects of tmCLIC1omab antibody in tumours.

The effects on tumours were mainly studied in brain tumours, in particular glioblastoma. Some results obtained with other types of tumour are also reported. It can be concluded that, albeit with different mode of action, the tmCLIC1omab antibody inhibits the growth and/or mobility of various solid tumours.

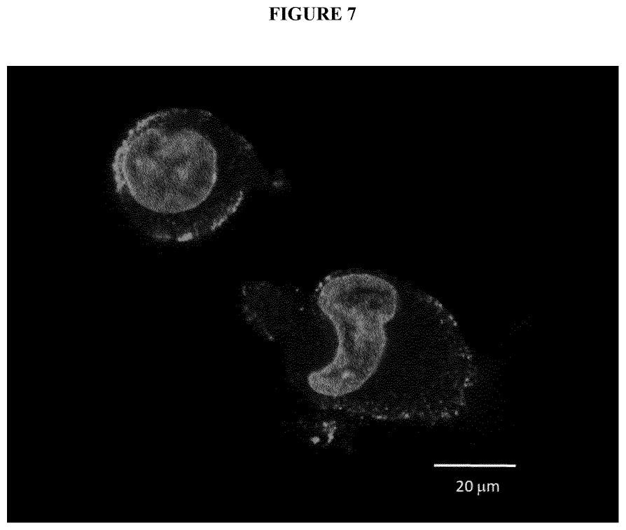

As in the case of microglial cells activated by β-amyloid, the distribution of tmCLIC1 is recognised by tmCLIC1omab (FIG. 7). Tumour cells are constitutionally activated cells which exhibit similar characteristics to microglial cells chronically stimulated by β-amyloid. Also in tumour cells, tmCLIC1 protein appears and stabilises in the membrane, with an increase in tmCLIC1-mediated ion current, major ROS production and a consequent exponential proliferation trend.

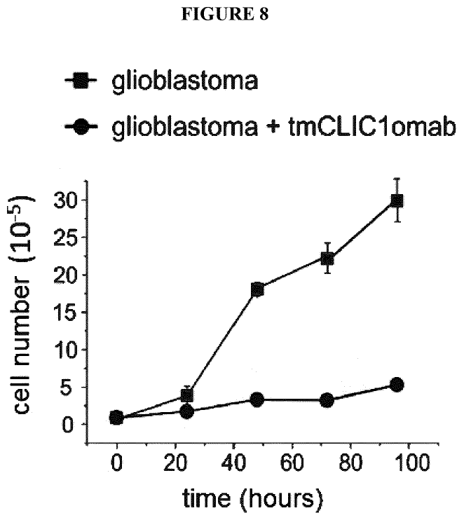

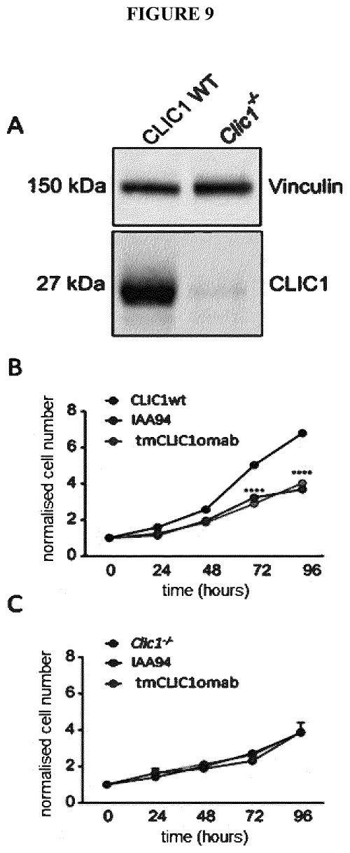

FIG. 8 shows the growth curve of a primary culture of human glioblastoma cells. Treatment with tmCLIC1omab inhibited cell growth in the period considered. A knockout cell line for CLIC1 protein (Clic1^{-/-}) was generated to monitor specificity. FIG. 9 shows a Western Blot that demonstrates the absence of CLIC1 protein in Oki′ cells (A), and three growth curves of glioblastoma cells, grown in parallel under control conditions, in the presence of tmCLIC1omab and in the presence of IAA94 (B), the specific tmCLIC1 blocker. It was also observed that Oki′ cells and cells wherein tmCLIC1 was pharmacologically inhibited by treatment with IAA94 or tmCLIC1 omab exhibit the same growth rate (C).

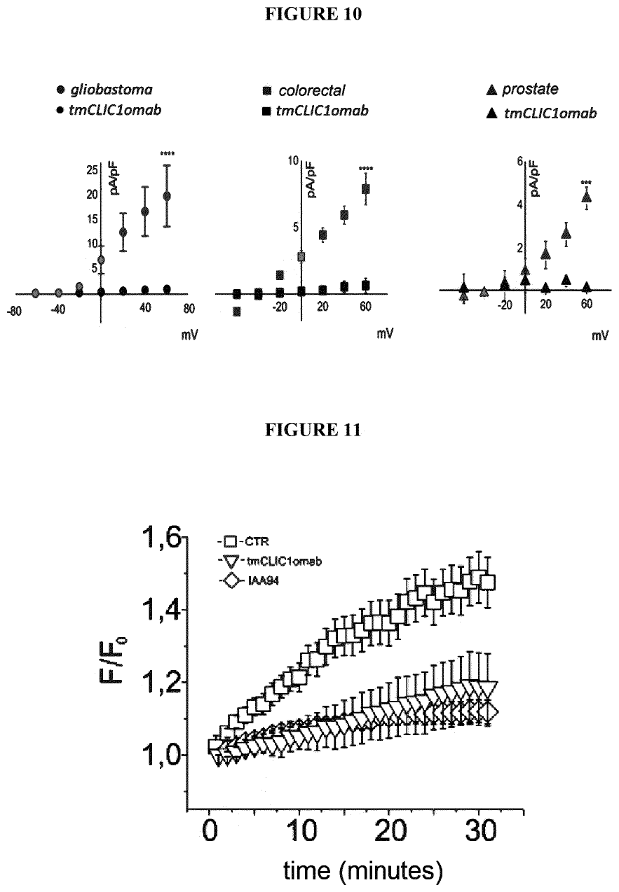

The functional experiments based on measuring the membrane ion current that passes through the tmCLIC1 ion channel were conducted on three different types of tumour cell. FIG. 10 shows three current/voltage relationship, indicating the membrane current values obtained with electrophysiological measurements. The data relate to glioblastoma, colorectal cancer and prostate cancer cells. The grey symbols relate to the current values in the control cells. In all three cases, the application of the tmCLIC1omab antibody depressed and almost eliminated the membrane current (black symbols).

In the subsequent functional experiment, ROS production was measured over time. The graph in FIG. 11 relates to the acquisition of fluorescence signals proportional to the amount of cytoplasmic ROS in glioblastoma cells. The cells were treated with both the antibody and the tmCLIC1 channel blocker. The fluorescence increase profile demonstrates that both treatments reduce the slope of the ROS increase curve about fivefold.

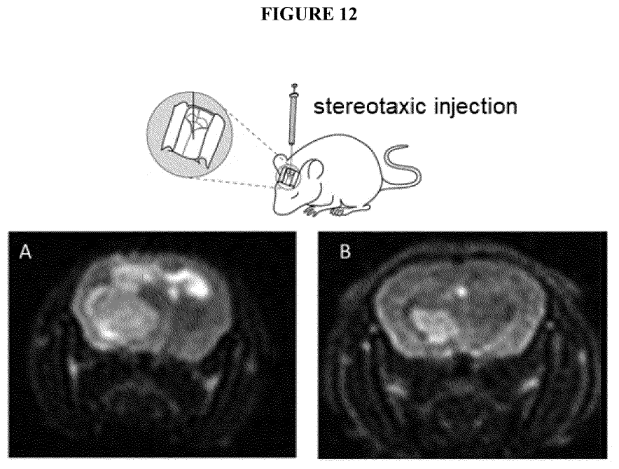

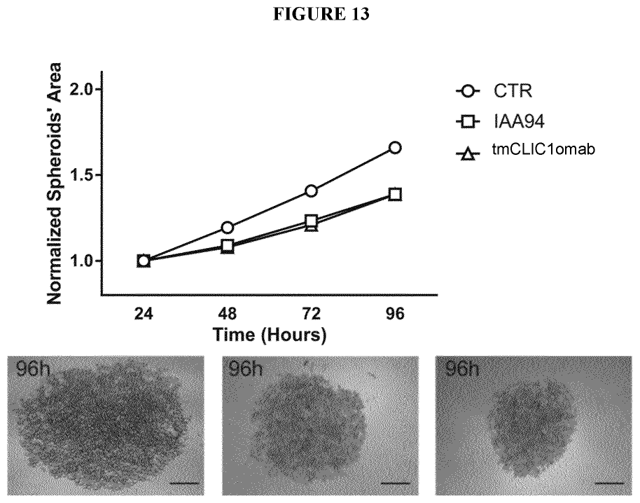

In the in vivo experiments, human glioblastoma cells were incubated for 72 hours with tmCLIC1omab antibody before being inoculated into the brain of immunodepressed mice. As shown in FIG. 12, the magnetic resonance scan performed two months after inoculation demonstrates that the control cells almost entirely filled a cerebral hemisphere (A). Conversely, the cells treated with the antibody possess a much smaller mass.

To evaluate whether tmCLIC1omab is able to penetrate across tumor spheroids, a 3D culture was performed. After the complete formation of spheroids, treatments were added and the area was measured every 24 hours up to 96 hours. The area at each time point was then normalized on the initial spheroids' area. As is visible in FIG. 5, also in 3D culture, both treatments were able to impair proliferation of spheroids significantly after 48 hours of incubation for both cell lines demonstrating the efficacy of IAA94 and antibody also in more structured complexes.

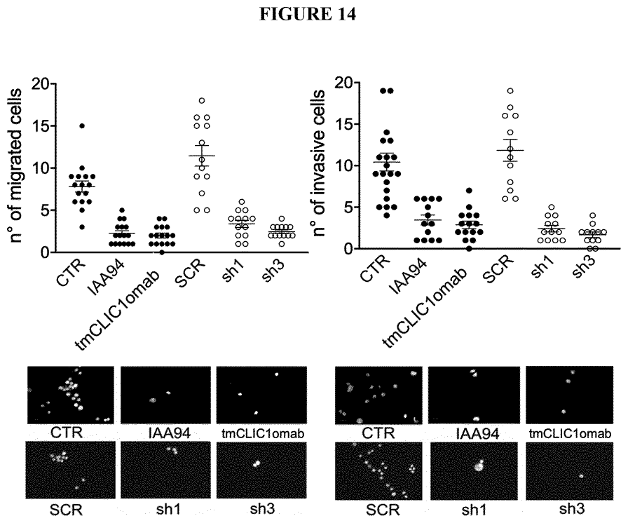

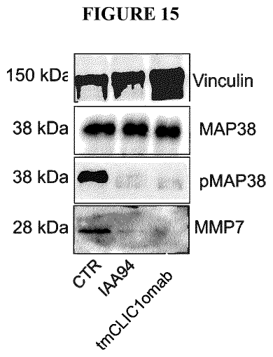

The overexpression of tmCLIC1 in the most aggressive cell lines of colorectal cancer and its pivotal role in regulation of cell cycle progression and proliferation, opens a question on the function of tmCLIC1 activity on migration and invasion potential of CRCs, that makes colorectal cancer still uncurable. We performed a Trans well assay to understand whether CLIC1 impairment could affect the migration and invasion potential of COLO201. Cells were plated after 60 hours of serum deprivation on a porous membrane, permeable only to growth factor, in contact to complete medium. The resulting chemo-attractant gradient leads the migrative cells to cross the membranes that are then stained with DAPI to visualize the nuclei. After 72 h of culture, as is depicted in FIG. 14, in both treated cells and CLIC1 knockdown cells, the migration potential is significantly impaired compared to controls. For invasion assays, we coated with Matrigel the porous membrane to mimic the extracellular matrix that cells must disrupt to invade tissues and mucosa. Also in this case, after 72 h of culture, it is visible that the number of invasive nuclei is significantly lower in treated and silenced cells compared to controls, demonstrating that CLIC1 impairment results in the block of invasive and migration potential. Considering that migration and invasion potential are regulated by a huge number of protein families, the effect of tmCLIC1 inhibition on the expression of two of the most representative proteins involved in invasion and migration of colorectal cancer cells was investigated, namely the Mitogen-Activated Protein Kinase 38 (MAP38) in its total and phosphorylated form (pMAP38), that was found to be extremely important for migration and overexpressed in the CRC lines, and the Metalloproteinase 7 (MMP7), found to be a key regulator of invasive mechanisms and overexpressed in COLO201. As shown in Figure in COLO201 the expression of the activated form of MAP38 (pMAP38) is significantly impaired compared to control. At the same time, also MMP7 expression is mainly abolished in the presence of tmCLIC1 inhibitors demonstrating that the alteration of tmCLIC1 activity has a consequence on the expression of the most important proteins involved in migration and invasion potential.

Previous Report of CLIC1 Antibody

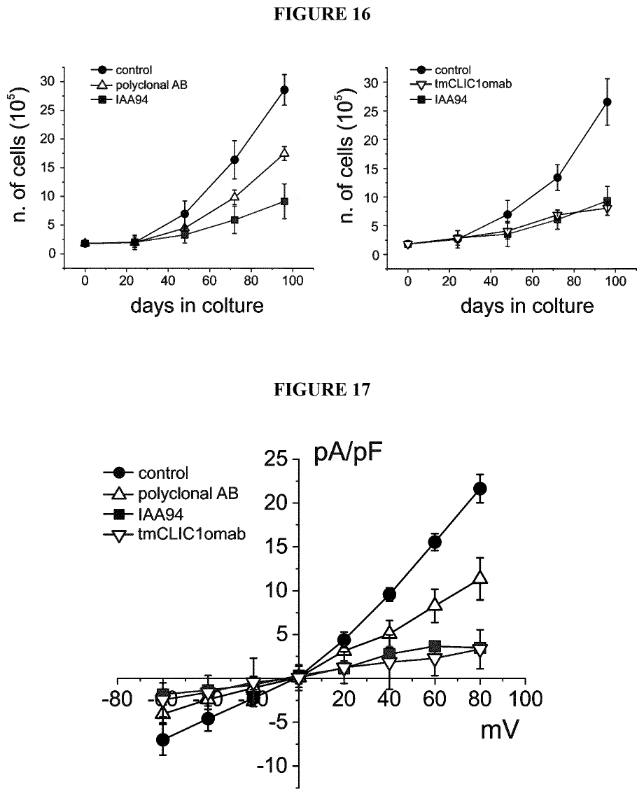

The paper by Averaimo et al. (J. of Neurochemistry 131, 444-456 (2014); doi: reports the use of a polyclonal antibody against the external portion of CLIC1 protein. The antibody is able to bind CLIC1 external portion in in vitro experiments. It recognizes the membrane protein and has a mild ability to impair CLIC1 ion channel function. FIG. 16 shows a comparison between mouse glioblastoma cells growth curves. It is evident that the polyclonal antibody is significantly less efficient than tmCLIC1omab compared to IAA94 action. This is evident also in acute application experiments. FIG. 17 shows a current/voltage relationship of CLIC1 ionic whole-cell current, elicited by voltage steps from −60 to +80 mV of the membrane potential. The maximum inhibition of CLIC1 ionic permeability operated by IAA94 added to the external solution can be matched only by tmCLIC1omab. The polyclonal antibody is much less efficient as a CLIC1 current inhibitor.

Materials and Methods

The reference sequence of the CLIC1 protein used in the experiments described in the present patent application is filed at UniProt under number UniProtKB—000299 (CLIC1 human), and described in the article Harrop S. J. et al., “Crystal structure of a soluble form of the intracellular chloride ion channel CLIC1 (NCC27) at 1.4-Å Resolution”; The Journal of Biological Chemistry, Vol. 276 No. 48, November 30, pp. 44993-45000 (2001).

The CLIC1 ion channel blocker used in the experiments and identified as IAA94 is the compound indanyloxyacetic acid 94, also known as R(+)-methylindazone or R(+)-[(6,7-dichloro-2-cyclopentyl-2,3-dihydro-2-methyl-1-oxo-1H-inden-5-yl)-oxy]acetic acid, of formula:

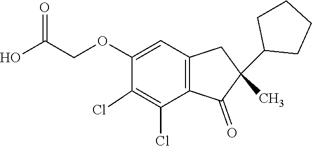 [SMILES: C[C@]1(C2CCCC2)Cc2cc(OCC(=O)O)c(Cl)c(Cl)c2C1=O]

Isolation of Antibodies

The specific antibodies for the NH2-CLIC1 molecule portion were obtained from four different mice. The resulting polyclonal antibodies were isolated after a 138-day immunisation protocol with a synthetic peptide conjugated with OVA (Ovalbumin), with sequence NH2-EQPQVELFVKAGSDGAKIGNC-COOH (SEQ ID NO.:21), corresponding to the sequence expressed in human tmCLIC1 protein extracellular portion. Said polyclonal antibodies were then isolated using the Protein G Sepharose technique, specifically designed to obtain IgG monoclonal antibodies. The specificity of the antibodies was demonstrated by the ELISA technique, and further verified by Western Blot.

Antibody Production

Total RNA was isolated from hybridoma cells according to the technical specifications of the RNeasy Plus Micro Kit (QIAGEN, Cat. No.: 74034). The total RNA was then converted to cDNA by reverse transcriptase using mouse-specific antisense primers, or alternatively by following the SMARTScribe Reverse Transcriptase technical manual (TaKaRa, Cat. No.: 639536). The heavy chain and light chain fragments of the antibody were then amplified according to the GenScript standard operating procedure (SOP) for rapid amplification of cDNA (RACE). The amplified fragments were then cloned separately with standard cloning vectors. PCR cloning was used to select clones with inserts of the correct size.

Cell Cultures

The primary glioblastoma cell lines, already tested for stem cell properties and carcinogenicity, were kindly supplied by Professor T. Florio's laboratory at Genoa University (Genoa, Italy). The cells were obtained from a surgical biopsy performed in the Neurosurgery Department of IRCCS-AOU San Martino IST (Genoa, Italy), from patients who had not received any treatment before the operation. The samples were histologically classified as GB grade IV (according to the WHO classification), and were used after obtaining the patients' informed consent and the approval of the Institutional Ethics Committee (IEC). The cells were cultured in stem cell-permissive medium consisting of Dulbecco's Modified Eagle's Medium (DMEM) and F12-GlutaMAX at the ratio of 1:1, supplemented with 1×B27 (Thermo Fisher Scientific), 10 μg/μL of fibroblast growth factor (FGF, Miltenyi Biotec), 20 μg/μL of human epidermal growth factor (EGF, Miltenyi Biotec) and 1% penicillin/streptomycin (Pen/Strep, 100 U/L) (Thermo Fisher Scientific).

Mesenchymal stem cells were obtained from post-caesarean section human umbilical cords at the Obstetrics and Gynaecology Department of Ospedale Evangelico Internazionale (Genoa, Italy), after obtaining informed consent and IEC approval. After removal of the blood vessels, the cords were treated with collagenase (0.5 μg/ml) to expose Wharton's jelly and obtain single cells. The cells were cultured in MesenPRO RS basal medium+Supplement (Thermo Fisher Scientific) after phenotypic characterisation by flow cytometry (MSC Phenotyping Kit, Miltenyi Biotec). Briefly, over 95% of cells tested negative for haematopoietic antigens (CD45, CD34, CD14) and MHC class II, and expressed CD73, CD105, CD90, CD29 and MHC class I.

Normal colon (CCD841) and cancerous colon (SW620) cell lines, and normal prostate (PNT2) and cancerous prostate (PC3) cell lines, were obtained from the IFOM cell bank (Milan, Italy). The colon cells were cultured in DMEM, and the prostate cells in RPMI. 10% foetal bovine serum (FBS, Euroclone) and 1% penicillin/streptomycin (Pen/Strep, 100 U/L) (Thermo Fisher Scientific) were added to both media.

The neurone cell cultures were obtained from the cerebral cortex of neonatal rats according to the pre-set protocol [7] and maintained in Neurobasal medium with the addition of B-27 Supplement (Thermo Fisher Scientific).

The microglial cells used (BV-2) originated from an immortalised cell line deriving from C57BL/6 mice and commonly marketed. The culture medium used was RPMI (Thermo Fisher Scientific) with the addition of 10% FBS (Euroclone) and 1% Pen/Strep (100 U/L, Thermo Fisher Scientific).

All the cell lines used were maintained at 37° C. 5% CO2.

Isolation of Lymphocytes and Monocytes from Peripheral Blood

Peripheral blood samples were taken, after obtaining informed consent and approval from the Ethics Committee, from control individuals of various age groups and individuals with a diagnosis of full-blown Alzheimer's disease at various stages.

For each individual, 8 ml of peripheral blood was collected in BD Vacutainer® CPT™ test tubes with sodium citrate, gently mixed by inversion and centrifuged at room temperature for 20 min. at 1600 rpm to separate three distinct phases: a bottom layer containing red blood cells, an intermediate layer containing mononuclear cells, and a top layer containing plasma. The layer of mononuclear cells was collected together with part of the plasma and transferred to a centrifuge tube. PBS was added until a final volume of ml was reached, and it was gently mixed by inversion and centrifuged for 10 min. at 300 rpm. The resulting pellet underwent two further washes in 5 ml of PBS, and was used for the subsequent experiments.

The pellet of mononuclear cells isolated from the blood was counted and divided into samples containing an equal number of cells (1×10⁶ cells/sample). Each sample was resuspended in 90 μl of cold staining solution (0.5% BSA, 2 mM EDTA in PBS) and, after the addition of 10 μl of FcR blocker (Miltenyi Biotec), was incubated in ice for 15 min. 10 μl of primary antibody diluted in staining solution was then added, and the sample was incubated for 1 h in ice. At the end of the incubation period, each sample was washed with 1 ml of cold staining solution and centrifuged at 350 rpm for 5 min. After two further washes, the cell pellet was resuspended in 100 μl of secondary antibody diluted in staining solution, and incubated for 30 min. in ice in the dark. 3 further washes in staining solution were performed, and the pellet was finally resuspended in 350 μl of PBS.

For the immunofluorescence experiment, the cell suspension was fixed for 10 min. in 2% paraformaldehyde (Sigma Aldrich), washed twice in PBS and incubated for 15 min. with DAPI diluted in PBS to a final concentration of 0.1 μg/mL. The samples were then centrifuged and resuspended in 30 μl of PBS. A drop of cell suspension was deposited on a slide and, after application of the mounting medium, covered with a coverslip. The resulting slides were then viewed under the microscope.

For the quantitative fluorescence analysis experiment, the cell suspension obtained was divided between the wells of a black 96-well plate (100 μl/well) and analysed with a microplate reader.

Immunofluorescence

Immunofluorescence analyses were conducted to evaluate the specificity of the antibody in binding the transmembrane portion of the CLIC1 protein in WT and Clic1^{-/-} cells. The cells (5×10⁴ cells/well) were plated on slides (ϕ12 mm). The cells were kept viable throughout the initial processes. The cells were washed three times with PBS Ca²⁺/Mg²⁺, and incubated for 45 minutes at room temperature in blocking solution (5% BSA in PBS). Subsequently, after removal of the blocking solution, the solution containing the antibody (3.5 μg/ml in PBS/BSA 5×) was added, and incubated for 1 h at room temperature. After three washes with PBS Ca²⁺/Mg²⁺, the cells were fixed with paraformaldehyde (PFA 2%) for 5 minutes at room temperature. After being washed three times with PBS Ca²⁺/Mg²⁺, the cells were incubated with secondary antibody Alexa Fluor 488 (Thermo Fischer Scientific) for 2 h at room temperature in the dark. After two washes with PBS Ca²⁺/Mg²⁺, the last wash was performed with PBS. The slides were incubated with DAPI to identify the nuclei (5 μg/ml in PBS) for 10 min, mounted on a slide with the use of Mowiol mounting medium, and stored in the dark at 4° C. The slides were observed through a Zeiss Examiner A1 fluorescence microscope with a Zeiss 40×/0.75 NA water immersion lens.

Quantitative Analysis of Fluorescence Intensity

This assay was conducted to evaluate the total amount of fluorescence signal associated with expression of tmCLIC1 in the control and Oki′ cell populations. The live cells (1×10⁶ cells/well) were washed three times and incubated in blocking solution (1.5% BSA in PBS) for 30 min. in ice. The antibody was added directly to the samples, in the presence of the blocking solution, and incubated for a further 2 h in ice. After washing, the samples were incubated with a solution containing secondary antibodies for 1 h in ice and in the dark. The samples were again washed three times and distributed on a black 96-well plate. All washes and the staining stages were conducted while maintaining the cells in suspension. The samples were analysed with an EnSight Multimode Plate Reader (PerkinElmer) using the appropriate filter to display the intensity of the fluorescence emitted by the Alexa Fluor 488 conjugated antibody (Em=488 nm; Ex=350 nm). The fluorescence intensity values of the samples incubated with anti-NH₂-CLIC1 are proportional to the amount of tmCLIC1, and were normalized to the values of the samples only incubated with secondary antibody.

Electrophysiological Recordings: Patch Clamp Technique

The microelectrodes were obtained from borosilicate capillaries with a diameter of 1.5 mm. Micropipettes were produced with a P-97 Brown-Flaming puller (Sutter Instruments, Novato, CA), with which the capillaries were forged to a tip diameter of 1-1.5 μm and electrical resistance of 5-8 MΩ.

The perforated patch clamp configuration was used for the whole-cell experiments, employing the antibiotic gramicidin (final concentration in pipette 5 μg/ml) which forms pores in the membrane that are only permeable to monovalent cations; in this way, the cytoplasmic concentration of the chloride ion remains unchanged throughout the experiment. The ion currents were digitised at 5 kHz and filtered at 1 kHz. Clampex 9.2 was used as the data acquisition and analysis programme.

In the time-course experiments, the potential was set on the basis of the resting potential of each cell, and a voltage step of +60 mV was applied every 5 seconds. The current measured is that observed at the end of the voltage step lasting 800 ms. When the amplitude of the current reaches a constant value, the antibody and IAA94 are added in sequence.

For the current-voltage graph, the voltage step protocol used to isolate the currents consisted of voltage variations ranging from −60 mV to +60 mV, with progressive increments of 20 mV lasting 800 ms. The potential was set on the basis of the resting potential of each cell (between 0 and −80 mV). The CLIC1-mediated chlorine currents were isolated from other ion currents by mathematical subtraction of the residual current following perfusion of 100 μM IAA94 dissolved in the external solution.

Growth Curves

The cells were seeded in 24-well plates (20,000 cells/well) and counted after 24, 48, 72 and 96 hours to construct the growth curve. The cells were collected and centrifuged, and the resuspended pellet was diluted 1:1 with Trypan Blue. An automated counter (Countess II, Thermo Fisher Scientific) was used to count the cells. All data were normalized to their controls.

Cell Viability Test

The cells were plated in 24-well multiwell plates at a density of 10,000 cells/cm 2. The number of live and dead cells for each sample was counted after 72 hours with an automated counter (Countess II, Thermo Fisher Scientific), following the same process as used for the growth curves. All data were standardised to the corresponding controls.

Flow Cytometry Cell Cycle Analysis

The cells were washed in PBS, fixed at 4° C. with 70% ethanol for 1 hour, and resuspended in the staining solution (20 mg/mL of RNase A, 50 μg/mL of propidium iodide, 0.5% of Triton X-100 in PBS, Sigma-Aldrich). The DNA content was quantified by FACScalibur (BD Bioscience). The cell cycle profile was determined by Listmode data analysis using ModFit LT software (Verity Software House). At least 10,000 events were collected by gating single nuclei and excluding the aggregates.

Time-Lapse Microscopy

For the proliferation experiments, the cells were plated in a 6-well plate (3×10⁵ cells/well). The time intervals were performed with a ScanR system (Olympus) based on an inverted IX81 microscope equipped with a Hamamatsu ORCA-Flash4 camera and driven by CellSens (Olympus) software, or an Eclipse TE200-E microscope (Nikon) equipped with a Cascade 11-512 (photometric) camera driven by Metamorph software. Throughout the observation process, the cells were kept in the microscope stage incubator (Okolab). The cells were analysed with a 20×LUCPlanFLN lens (NA 0.45) or a 10× PlanFluor lens (NA 0.30). The images were acquired every 15 minutes for 36 hours, and analysed with Fiji software.

In Vivo Experiments

Patient-derived neurospheres were mechanically dissociated, resuspended in 2 of PBS, and injected into the caudate nucleus (1 mm posterior, 3 mm left lateral, 3.5 mm deep relative to bregma) of 5-week-old female CD1 nu/nu mice (Charles River, Wilmington, MA). The cells were incubated with the antibody or the control isotype for 72 hours before implantation (10⁵ cells) into the brain of immunodeficient mice. The experiments were conducted pursuant to Italian law (Order of the Executive 116/92, as amended), which transposes EU Directive 86/609 (Council Directive 86/609/EEC of 24 Nov. 1986 on the approximation of laws, regulations and administrative provisions of the Member States regarding the protection of animals used for experimental and other scientific purposes).

Spheroids' Formation

Spheroids were formed with hanging-drop method and then plated in rounded 96 plates coated with 5% of agar. After the complete formation of spheroids, treatments were added and the area was measured every 24 hours up to 96 hours. The area at each time point was then normalized on the initial spheroids' area.

Migration and Invasion Assays

Cells were plated after 60 hours of serum deprivation on a 0.22 μM porous membrane, permeable only to growth factor, in contact to complete medium. Membranes were cut, stained with DAPI dye, and included with mowiol. Cover glasses were examined under Zeiss Examiner A1 fluorescence microscope with a Zeiss 40×/0.75 NA water immersion lens.

Mapping

PEPperMAP epitope Mapping® of the tmCLIC1omab antibody was conducted against the peptide EQPQVELFVKAGSDGAKIGNC (amino acids 3-24 of the CLIC1 protein) translated to linear peptides consisting of 5, 10 and 15 amino acids with peptide-peptide overlaps of 4, 9 and 14 amino acids for high-resolution epitope data. The resulting CLIC1 peptide microarrays were incubated with the antibody samples at concentrations of 1 μg/ml, 10 μg/ml, 100 μg/ml and 250 μg/ml in incubation buffer, followed by staining with secondary and control antibodies and reading with a LI-COR Odyssey Imaging System. The intensity of the points and peptide annotation were quantified with the PepSlide® Analyzer.

Pre-staining of a copy of a microarray of CLIC1 peptides with the secondary antibody did not indicate any background interaction with the CLIC1 linear peptides that might interfere with the main assays.

Conversely, incubation of a second copy of the microarray of CLIC1 peptides with the tmCLIC1omab monoclonal antibody gave an antibody response with the epitopes formed by peptides adjacent to the VELF consensus motif (SEQ ID NO:22), with all peptide lengths having low or moderate signal-to-noise ratios. Additional very weak interactions of the antibody with the HA control peptides were observed, perhaps because of a cross-reaction or a less specific antibody bond. With the peptides consisting of 10 and amino acids, longer GEQPQVELF (10 aa) (SEQ ID NO:23) and SGSGSGEQPQVELF (15 aa) (SEQ ID NO:24) consensus motifs were observed, the underlined letters of which form part of linker GSGSGSGSG (SEQ ID NO:25) specific to the microarray. In conclusion, the antibody exhibits specificity with the QVELF sequence (SEQ ID NO:26) and, albeit with lower affinity, binds the QVELFV sequence (SEQ ID NO:27) well.

### REFERENCES

1. Littler, D. R., et al., The intracellular chloride ion channel protein CLIC1 undergoes a redox-controlled structural transition. J Biol Chem, 2004. 279(10): p. 9298-305.
2. Harrop, S. J., et al., Crystal structure of a soluble form of the intracellular chloride ion channel CLIC1 (NCC27) at 1.4-A resolution. J Biol Chem, 2001. 276(48): p. 44993-5000.
3. Warton, K., et al., Recombinant CLIC1 (NCC27) assembles in lipid bilayers via a pH-dependent two-state process to form chloride ion channels with identical characteristics to those observed in Chinese hamster ovary cells expressing CLIC1. J Biol Chem, 2002. 277(29): p. 26003-11.
4. Valenzuela, S. M., et al., The nuclear chloride ion channel NCC27 is involved in regulation of the cell cycle. J Physiol, 2000. 529 Pt 3: p. 541-52.
5. Tonini, R., et al., Functional characterization of the NCC27 nuclear protein in stable transfected CHO-KJ cells. FASEB J, 2000. 14(9): p. 1171-8.
6. Carlini, V., et al., CLIC1 Protein Accumulates in Circulating Monocyte Membrane during Neurodegeneration. Int J Mol Sci, 2020. 21(4).
7. Novarino, G., et al., Involvement of the intracellular ion channel CLIC1 in microglia-mediated beta-amyloid-induced neurotoxicity. J Neurosci, 2004. 24(23): p. 5322-30.
8. Averaimo S, Gritti M, Barini E, Gasparini L, Mazzanti M. (2014). CLIC1 functional expression is required for cAMP-induced neurite elongation in postnatal mouse retinal ganglion cells. J Neurochem. November; 131(4):444-56.

1. An antibody against the extracellular portion of the CLIC1 human protein in its plasma membrane location, said antibody being able to inhibit the chloride ion current mediated by CLIC1 by at least 90% compared with the inhibition caused by the IAA94 CLIC1 channel blocker at a concentration of 100 wherein said chloride current is measured by the patch clamp technique in whole-cell configuration on a single glioblastoma cell or in a colorectal cancer cell line, and said IAA94 channel blocker has the following structural formula:

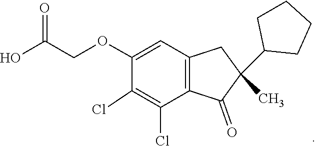 [SMILES: C[C@]1(C2CCCC2)Cc2cc(OCC(=O)O)c(Cl)c(Cl)c2C1=O]

2. Antibody according to claim 1, which recognises a CLIC1 epitope containing the amino acid sequence QVELF (SEQ ID NO:26).

3. Antibody according to claim 1, which comprises a heavy chain variable domain (VH) and a light chain variable domain (VL), both of which domains in turn comprise three Complementary Determining Regions (CDRs), wherein:
VH CDR1, CDR2 and CDR3 comprise sequences SEQ ID NO:1, SEQ ID NO:2 and SEQ ID NO:3 respectively, or sequences at least 90%, identical thereto;
and
VL CDR1, CDR2 and CDR3 comprise sequences SEQ ID NO:4, SEQ ID NO:5 and SEQ ID NO:6 respectively, or sequences at least 90%, identical thereto;
or
VH CDR1, CDR2 and CDR3 comprise or consist of the sequences SEQ ID NO:7, SEQ ID NO:8 and SEQ ID NO:9 respectively, or sequences at least 90%, identical thereto; and
VL CDR1, CDR2 and CDR3 comprise sequences SEQ ID NO:10, SEQ ID NO:11 and SEQ ID NO:12 respectively, or sequences at least 90%, identical thereto;

4. Antibody according to claim 3, wherein:
the heavy chain variable domain (VH) comprises sequence SEQ ID NO:13, or a sequence at least 90%, identical thereto;
and
the light chain variable domain (VL) comprises sequence SEQ ID NO:14, or a sequence at least 90%, identical thereto;
or
the heavy chain variable domain (VH) comprises sequence SEQ ID NO:15, or a sequence at least 90%, identical thereto; and
the light chain variable domain (VL) comprises sequence SEQ ID NO:16, or a sequence at least 90%, identical thereto.

5. Antibody according to claim 1, which is an immunoglobulin G of IgG1, IgG2, IgG3 or IgG4 subclass.

6. A fragment of an antibody according to claim 1, which is selected from Fab, Fab′, Fab′-SH, Fv, scFv, (Fab′)2, diabodies (dAb) or single domain antibody (sdAb).

7. A nucleic acid molecule encoding an antibody according to claim 1 or a fragment thereof which is selected from Fab, Fab′, Fab′-SH, Fv, scFv, (Fab′)2, diabodies (dAb) or single domain antibody (sdAb).

8. An expression vector comprising a nucleic acid molecule according to claim 7, or a host cell comprising said vector.

9. A diagnostic or therapeutic composition comprising an antibody according to claim 1 or a fragment thereof which is selected from Fab, Fab′, Fab′-SH, Fv, scFv, (Fab′)2, diabodies (dAb) or single domain antibody (sdAb).

10. Method of diagnosing or treating inflammation, neurodegenerative diseases or solid tumours in patients in need thereof with the antibody according to claim 1, or a fragment thereof selected from Fab, Fab′, Fab′-SH, Fv, scFv, (Fab′)2, diabodies (dAb) or single domain antibody (sdAb) or a composition thereof, said method comprising:
administering said antibody, said fragment thereof or said composition thereof to said patients.

11. The method according to claim 10, wherein said neurodegenerative disease is Alzheimer's disease.

12. The method according to claim 10, wherein said solid tumours are brain cancer, glioblastoma, colorectal, pancreatic, prostate or breast cancer.
